(19) United States
(12) Patent Application Publication
(10) Pub. No.: US 2010/0086586 A1
(43) Pub. Date: Apr. 8, 2010

(54) Pharmaceutical Composition

(76) Inventors: Brett Antony Mooney, Mt. Ommaney (AU); Erwin Owusu-Gyamfi, Herts (GB)

Correspondence Address: SUMMA, ADDITON & ASHE, P.A., 11610 NORTH COMMUNITY HOUSE ROAD, SUITE 200, CHARLOTTE, NC, 28277, US

(21) Appl. No.: 12/520,409

(22) PCT Filed: Dec. 21, 2007
(86) PCT No.: PCT/AU2007/001998

§ 371 Date: Dec. 1, 2009

(30) Foreign Application Priority Data
Dec. 21, 2006 (AU) 2006907173

(51) Int. Cl. A61K 9/48; A61K 31/585; A61K 9/14; A61K 9/20; A61K 9/28; A61P 9/10; A61P 9/12

(52) U.S. Cl. 424/451; 514/173; 424/489; 424/464; 424/474

(57) ABSTRACT

A pharmaceutical composition comprising eplerenone having a D90 particle size of between 15-25 microns and further comprising one or more pharmaceutically acceptable excipients.

### TECHNICAL FIELD

The present invention relates to pharmaceutical compositions comprising the 9,11α-epoxy steroid derivative, eplerenone as active ingredient. Further, the invention relates to the use of such compositions in the manufacture of medicaments for the treatment of conditions for which eplerenone is effective and processes to manufacture these compositions. The invention also relates to eplerenone of a specific particle size.

### BACKGROUND ART

The compound (7α,11α,17α)-9,11-Epoxy-17-hydroxy-3-oxopregn-4-ene-7,21-dicarboxylic acid γ-lactone methyl ester, hereinafter referred to as eplerenone and also known as 9α,11-epoxy-7α-(methoxycarbonyl)-3-oxo-17α-pregn-4-ene-21,17-carbolactone or 9α,11α-epoxy-7α-methoxy-carbonyl-20-spirox-4-ene-3,21-dione or epoxymexrenone was first disclosed in U.S. Pat. No. 4,559,332 (assigned to Ciba Geigy). Eplerenone is an aldosterone receptor antagonist having the structure:

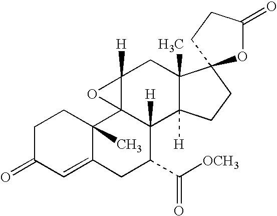

Eplerenone is indicated in addition to standard therapy including beta-blockers, to reduce the risk of cardiovascular mortality and morbidity in stable patients with left ventricular dysfunction (LVEF≦40%) and clinical evidence of heart failure after recent myocardial infarction. Additionally, it is indicated for the treatment of hypertension alone or in combination with other anti-hypertensive agents.

EP 122232 A/U.S. Pat. No. 4,559,332 both assigned to Ciba Geigy and incorporated herein by reference, relates to novel 20-spiroxanes and analogues. General references to enteral, including tablets and aqueous solutions, and parenteral formulations of eplerenone (an exemplary compound), are disclosed therein.

EP 1175220 B1 (Novartis) discloses compositions comprising eplerenone having a D90 particle size of less than 15 microns. The claimed advantages of such a composition include improved solubility of the composition, improved bioavailability, improved safety, improved dissolution profile for controlled release oral dosage forms and decreased dissolution time for immediate release oral dosage forms amongst others. It is also stated therein that similar compositions comprising eplerenone having a larger particle size are not as bioavailable as the disclosed compositions. The most preferred embodiments comprise eplerenone having a D90 particle size of less than 400 nm.

The problem with compositions comprising such small particle sizes is that there are some instances in which particle size reduction fails to increase absorption rate. One reason might be that dissolution is not the rate limiting step. Additionally micronisation sometimes increases the tendency of the particles to aggregate which may lead to a decrease in surface area. Further it has been reported that extremely small sizes may be inadvisable for some drug substances as adsorbed air or crystal growth might act as dissolution rate limiting steps. Thus, to sum up the effect on absorption behaviour for particle size reduction to extremely small particles less than 10 microns cannot be reliably predicted. The micronisation process itself can also lead to degradation of the active ingredient.

Conversely, relatively larger particle sizes of drugs that have low aqueous solubility can suffer from the problem of poor dissolution and consequently poor bioavailability. Thus, there is a need for compositions of eplerenone to provide improved or effective compositions that keep the beneficial properties of micronised particles, such as an increase in aqueous solubility, leading to an increase in bioavailability whilst overcoming the above highlighted problems of the prior art.

### SUMMARY OF THE INVENTION

The inventors have surprisingly found that, against the teaching of the prior art, a pharmaceutical composition comprising eplerenone of a defined particle size affords suitable properties which overcome the above problems associated with the prior art.

Accordingly there is provided in a first aspect of the invention a pharmaceutical composition comprising eplerenone having a D90 particle size of approximately 15-25 microns and further comprising one or more pharmaceutically acceptable excipients. In a preferred embodiment, the composition is provided wherein the D90 particle size diameter is approximately 17-23 microns, most preferably D90 particle size is approximately 20 microns.

In another particularly preferred embodiment, the composition according to the invention is a tablet composition. Preferably the tablet is coated. In an alternative embodiment the composition is a capsule.

Preferred embodiments of a composition according to the invention comprise eplerenone present in an amount of approximately 1-90% by weight of the composition.

Further embodiments of a composition according to the invention further comprise wetting agents/surfactants, preferred embodiments comprise Tween® (polysorbate) or particularly preferred is sodium lauryl sulphate.

The pharmaceutical composition of the first aspect of the invention can optionally include one or more additional API's. Preferably the API's are selected from the group comprising anti-arrhythmia's, anti-anginal and other treatments for hypertension and cardiovascular conditions and/or diseases.

A second aspect of the invention is the use of a composition according to the invention to treat hypertension or alternatively for the manufacture of a medicament to reduce the risk of cardiovascular mortality and morbidity in stable patients with left ventricular dysfunction (LVEF≦40%) and clinical evidence of heart failure after recent myocardial infarction.

A third aspect of the invention provides a method of treating hypertension or alternatively treating stable patients with left ventricular dysfunction (LVEF≦40%) and clinical evidence of heart failure after recent myocardial infarction to reduce the risk of cardiovascular mortality and morbidity, comprising administering a composition of the invention.

In a fourth aspect of the invention provides particulate eplerenone having a D90 particle size of between 15-25 microns, preferably between 17-23 microns, most preferably having a D90 particle size of approximately 20 microns. In a particularly preferred embodiment particulate eplerenone may be used to prepare pharmaceutical compositions according to the invention.

In a fifth aspect of the invention there is provided a process to prepare a pharmaceutical composition according to the invention comprising eplerenone having a particle size comprising admixing said eplerenone with one or more pharmaceutically acceptable carriers, preferably the composition is prepared by a process comprising wet or dry granulation techniques. A particularly preferred process comprises
i) admixing the particulate eplerenone with one or more pharmaceutical excipients.
ii) forming a wet granulation mixture
iii) granulating mixture
iv) dry granules
v) compress granules into tablet form
vi) optionally coating the tablet composition.

A sixth aspect according to the invention provides a process for preparing particulate eplerenone according to the invention comprising subjecting eplerenone to a technique chosen from the list comprising conventional comminution and de-agglomeration, micro-fluidisation and chemical means. Preferably the comminution techniques comprise grinding or milling in an air-jet mill or impact mill, a ball mill, vibration mill, mortar mill or pin mill. In further embodiments the chemical means comprises controlled precipitation/recrystallisation.

Of course it will be recognised by the skilled person that the compositions and eplerenone as disclosed herein lend themselves to a number of formulation types. For example controlled release compositions are within the scope of the invention. Such controlled-release compositions may comprise sustained release, delayed-release, modified-release. Further embodiments may also comprise multi-phasic release compositions wherein a proportion of the eplerenone is released immediately and release of the remainder is delayed. Further embodiments the composition may comprise additional API's with differing release kinetics.

### DESCRIPTION OF THE INVENTION

According to the first aspect of the invention there is provided a pharmaceutical composition comprising eplerenone have a D90 particle size diameter of between 15 and 25 microns. In order to produce eplerenone particles, e.g. crystals having the desired particle size and particle size distribution, conventional comminution and de-agglomeration techniques may be used, for example grinding in an air-jet mill or impact mill, a ball mill, vibration mill, mortar mill or pin mill. Further techniques such as micro-fluidisation can also be used. Chemical techniques such as controlled precipitation/recrystallisation may also be employed.

The known particle size analysis methods are suitable for determining the median particle size, for example particle size measurement using light, for example light-scattering methods or turbidimetric methods, sedimentation methods, for example pipette analysis using an Andreassen pipette, sedimentation scales, photosedimentometers or sedimentation in a centrifugal force field, pulse methods, for example using a Coulter counter, or sorting by means of gravitational or centrifugal force. Those methods are described, inter alia, in Voigt, loc. cit., pages 64-79.

The composition according to the invention may contain pharmaceutically acceptable excipients commonly used in pharmaceutical compositions, e.g. for oral administration.

In a preferred embodiment according to the invention the composition may be in the form of a tablet which comprises, a) a tablet core comprising a therapeutically effective dose of the eplerenone, preferably in a finely ground form, having a D90 particle size of approximately from 15-25 μm, preferably 17 to 23 μm, most preferably 20 μm and further excipients that are suitable for the manufacture of the compositions according to the invention.

A particularly preferred composition according to the invention comprises a tablet composition. Tablets according to the present invention comprise eplerenone of fine particle size and narrow particle size distribution and as such may be formulated into dosage forms, e.g. solid oral dosage forms such as the preferred tablets with relative ease. Furthermore, the fine particle size and narrow particle size distribution may also be beneficial in improving the bioavailability of eplerenone whilst still avoiding the problems that can be associated with fine particle sizes and that are prevalent in the prior art. Still further the compositions meet all customary requirements, such as storage stability and colour stability.

Tablets according to the invention may be manufactured by any means at the disposal of the skilled practitioner. Commonly used means include compressing eplerenone with conventional tabletting excipients to form a tablet core using conventional tabletting processes. Optionally the tablet cores may be coated. Coatings may comprise one or more of enteric release coatings, coatings that effect the release kinetics of eplerenone and conventional immediate release coatings for example the Opadry® series of aqueous film-coatings systems manufactured by Colorcon.

The tablet cores may be produced using conventional methods known in the art for example granulation methods, such as wet or dry granulation, with optional comminution of the granules and with subsequent compression and coating. Granulation methods are described, for example, in Voigt, loc. cit., pages 156-169.

Suitable excipients for the production of granules are, for example pulverulent fillers optionally having flow-conditioning properties, for example talcum, silicon dioxide, for example synthetic amorphous anhydrous silica acid of the Syloid® X type (Grace), for example SYLOID® 244 FP, microcrystalline cellulose, for example the Avicel® types (FMC Corp.) such as AVICEL® PH101, 102, 105, RC581 or RC 591, Emcocel® type (Mendell Corp.) or Elcema type (Degussa); carbohydrates, such as sugars, sugar alcohols, starches or starch derivatives, for example lactose, dextrose, saccharose, glucose, sorbitol, mannitol, xylitol, potato starch, maize starch, rice starch, wheat starch or amylopectin, tricalcium phosphate, calcium hydrogen phosphate or magnesium trisilicate; particularly preferred is microcrystalline cellulose; binders, such as gelatin, tragacanth, agar, alginic acid, cellulose ethers, for example methylcellulose, carboxymethylcellulose or hydroxypropyl methylcellulose, polyethylene glycols or ethylene oxide homopolymers, especially having a degree of polymerisation of approximately from 2.0×103 to 1.0×105 and an approximate molecular weight of about from 1.0×105 to 5.0×106, for example excipients known by the name Polyoxe® (Union Carbide), polyvinylpyrrolidone or povidones, especially having a mean molecular weight of approximately 1000 and a degree of polymerisation of approximately from 500 to 2500, and also agar or gelatine particularly preferred binder is hydroxypropyl methyllcellulose such as Hypromellose®; surface-active substances, for example anionic surfactants of the alkyl sulphate type, for example sodium, potassium or magnesium n-dodecyl sulphate, n-tetradecyl sulphate, n-hexadecyl sulphate or n-octadecyl sulphate, of the alkyl ether sulphate type, for example sodium, potassium or magnesium n-dodecyloxyethyl sulphate, n-tetradecyloxyethyl sulphate, n-hexadecyloxyethyl sulphate or n-octadecyloxyethyl sulphate, or of the alkanesulfonate type, for example sodium, potassium or magnesium n-dodecanesulfonate, n-tetradecanesulfonate, n-hexadecanesulfonate or n-octadecanesulfonate, or non-ionic surfactants of the fatty acid polyhydroxy alcohol ester type, such as sorbitan monolaurate, monooleate, monostearate or monopalmitate, sorbitan tristearate or trioleate, polyoxyethylene adducts of fatty acid polyhydroxy alcohol esters, such as polyoxyethylene sorbitan monolaurate, monooleate, monostearate, monopalmitate, tristearate or trioleate, polyethylene glycol fatty acid esters, such as polyoxyethyl stearate, polyethylene glycol 400 stearate, polyethylene glycol 2000 stearate, especially ethylene oxide/propylene oxide block polymers of the Pluronics® (BWC) or Synperonic® (ICI) type.

Granules may be produced in a manner known per se, for example using wet granulation methods known for the production of “built-up” granules or “broken-down” granules.

Methods for the formation of built-up granules may operate continuously and comprise, for example simultaneously spraying the granulation mass with granulation solution and drying, for example in a drum granulator, in pan granulators, on disc granulators, in a fluidised bed, by spray-drying or spray-solidifying, or operate discontinuously, for example in a fluidised bed, in a batch mixer or in a spray-drying drum.

Preferred are methods for the production of broken-down granules, which may be carried out discontinuously and in which the granulation mass first forms a wet aggregate with the granulation solution, which aggregate is then comminuted or formed into granules of the desired particle size and the granules then being dried. Suitable equipment for the granulation step are planetary mixers, low and high shear mixers, wet granulation equipment including extruders and spheronisers include, for example, apparatus from the companies Ligue, Glatt, Diosna, Fielder, Collette, Alexanderwerk, Ytron, Werner & Pfleiderer, Fuji, Mica, Caleva and Gabler.

The granulation mass consists of comminuted, preferably ground, eplerenone and the excipients mentioned above, for example pulverulent fillers, such as microcrystalline cellulose of the AVICEL® type. AVICEL® PH 102 is especially suitable, or wetting agents/surfactants. Tween® (polysorbate) or alternatively sodium lauryl sulphate is a particularly preferred. surfactant. Depending on the method used, the granulation mass may be in the form of a premix or may be obtained by mixing the eplerenone into one or more excipients or mixing the excipients into the eplerenone. The wet granules are preferably dried, for example in the described manner by tray drying in an oven or drying in a fluidised bed dryer.

According to an alternative process variant, tablet cores are produced using the so-called compacting or dry granulation method in which the active ingredient is compressed with the excipients to form relatively large mouldings, for example slugs or ribbons, which are comminuted by grinding, and the ground material is compressed to form tablet cores.

Suitable excipients for the compacting method are preferably those which are suitable for the conventional direct compression methods, for example dry binders, such as starches, for example potato, wheat and maize starch, microcrystalline cellulose, for example commercial products available under the trademarks Avicel®, Hewetene® or Pharmacel®, highly dispersed silicon dioxide, for example Aerosil®, mannitol, lactose, and also polyethylene glycol, especially having a molecular weight of from 4000 to 6000, cross-linked polyvinylpyrrolidone (Polyplasdones XL or Kollidone® CL), cross-linked carboxymethylcellulose (Acdisol® X CMC-XL), carboxymethylcellulose [Nymcel, for example ZSB-10, (Nyma)], hydroxypropyl methylcellulose, for example the quality HPMC 603, carboxymethyl starch <RTI [Explotab® X (Mendell) or Primojel® (Scholtens)], microcrystalline cellulose, for example Avicel® PH 102, dicalcium phosphate, for example Emcompress® or talcum. The addition of small amounts of, for example, lubricants, such as magnesium stearate, is also advantageous.

Compression to form tablet cores may be carried out in conventional tabletting machines, for example EK-0 Korsch eccentric tabletting machines or rotary tabletting machines. The tablet cores may be of various shapes, for example round, oval, oblong, cylindrical etc., and various sizes, depending on the amount of eplerenone.

### EXAMPLES

The following Examples illustrate the invention, but in no way limit the scope of the invention. Further, the eplerenone in the examples below has been micronised using standard techniques known in the art and as described above to a particle size of between 15 and 25 microns. Preferably the D90 particle size is about 20 microns.

### Example 1

The ingredients of the pharmaceutical composition according to the invention can be prepared in accordance with acceptable pharmaceutical manufacturing practices. Preferably the manufacturing process will comprise wet granulation for example as described above, because of the amount of active pharmaceutical ingredient (API) required and also the lower compressibility of material at the preferred particle size.

An exemplary immediate-release composition according to the invention is shown in Table 1.

A 25 mg immediate release composition was prepared according to the following:

TABLE 1

| Ingredient | Mg |
|---|---|
| Eplerenone | 25 |
| Lactose Monohydrate 300# | 60 |
| Microcrystalline Cellulose 101 | 14 |
| Maize Starch | 8 |
| Povidone K30 | 4 |
| Polysorbate 80 | 1 |

### Example 2

A 50 mg immediate release composition was prepared according to the following:

TABLE 2

| Ingredient | Mg |
|---|---|
| Eplerenone | 50 |
| Lactose Monohydrate 300# | 120 |
| Microcrystalline Cellulose 101 | 28 |
| Maize Starch | 16 |
| Povidone K30 | 8 |
| Polysorbate 80 | 2 |

### Example 3

A 50 mg immediate release composition was prepared according to the following:

TABLE 3

| Ingredient | Mg |
|---|---|
| Eplerenone | 100 |
| Lactose Monohydrate | 240 |
| Microcrystalline Cellulose | 56 |
| (intragranular) | |
| Croscarmellose Sodium | 32 |
| Hydroxypropyl Methylcellulose | 16 |
| Microcrystalline Cellulose | 4 |
| (extragranular) | |

### Example 4

A coated 25 mg tablet composition was prepared according to the following:

TABLE 4

| Ingredient | Mg |
|---|---|
| Eplerenone | 25 |
| Lactose Monohydrate | 60 |
| Microcrystalline Cellulose | 14 |
| (intragranular) | |
| Croscarmellose Sodium | 8 |
| Hydroxypropyl Methylcellulose | 4 |
| Microcrystalline Cellulose | 1 |
| (extragranular) | |
| Opadry white | 3 |

### Example 5

A coated 50 mg tablet composition was prepared according to the following:

TABLE 5

| Ingredient | Mg |
|---|---|
| Eplerenone | 50 |
| Lactose Monohydrate | 120 |
| Microcrystalline Cellulose | 28 |
| (intragranular) | |
| Croscarmellose Sodium | 16 |
| Hydroxypropyl Methylcellulose | 8 |
| Microcrystalline Cellulose | 2 |
| (extragranular) | |
| Opadry white | 6 |

### Example 6

A coated 100 mg tablet composition was prepared according to the following:

TABLE 6

| Ingredient | Mg |
|---|---|
| Eplerenone | 100 |
| Lactose Monohydrate | 240 |
| Microcrystalline Cellulose | 56 |
| (intragranular) | |
| Croscarmellose Sodium | 32 |
| Hydroxypropyl Methylcellulose | 16 |
| Microcrystalline Cellulose | 4 |
| (extragranular) | |
| Opadry white | 12 |

### Example 7

A controlled release 25 mg tablet composition was prepared according to the following:

TABLE 7

| Ingredient | Mg |
|---|---|
| Eplerenone | 25 |
| Povidone K90 | 4 |
| PVA & PVP | 52.5 |
| Hydrogenated Vegetable Oil | 22 |
| Magnesium Stearate | 1 |

Of course it will be apparent to one skilled in the art that the above compositions can be modified as required for example by the inclusion of colorants or taste enhancers.

1. A pharmaceutical composition comprising eplerenone having a D90 particle size of between 15-25 microns and further comprising one or more pharmaceutically acceptable excipients.

2. A pharmaceutical composition according to claim 1 wherein the D90 particle size diameter is approximately between 17-23 microns.

3. A pharmaceutical composition according to claim 2 wherein the D90 particle size is approximately 20 microns.

4. A pharmaceutical composition according to claim 1 wherein the composition is a tablet composition.

5. A pharmaceutical composition according to claim 4 wherein the tablet is coated.

6. A pharmaceutical composition according to claim 1 which has been prepared by wet granulation.

7. A pharmaceutical composition according to claim 1 wherein the pharmaceutical composition is capsule composition.

8. A pharmaceutical composition according to claim 1 wherein the eplerenone is present in an amount of approximately 1-90% by weight of the composition.

9. (canceled)

10. A method of treating hypertension in a patient in need of such treatment comprising administering a pharmaceutical composition according to claim 1.

11. (canceled)

12. A method of reducing the risk of cardiovascular mortality and morbidity in stable patients with left ventricular dysfunction (LVEF≦40%) and clinical evidence of heart failure after recent myocardial infarction in a patient in need of such treatment comprising administering a pharmaceutical composition according to claim 1.

13. Particulate eplerenone having a D90 particle size of between 15-25 microns.

14. Particulate eplerenone according to claim 13 having a D90 particle size of between 17-23 microns.

15. Particulate eplerenone according to claim 13 having a D90 particle size of approximately 20 microns.

16. A process for preparing particulate eplerenone according to claim 13 comprising subjecting eplerenone to a technique chosen from the list comprising conventional comminution and de-agglomeration, micro-fluidisation and chemical means.

17. A process according to claim 16 wherein the comminution techniques comprise grinding in an air-jet mill or impact mill, a ball mill, vibration mill, mortar mill or pin mill.

18. A process according to claim 16 wherein the chemical means is selected from the group consisting of controlled precipitation or controlled recrystallization.

19. (canceled)

20. (canceled)

21. (canceled)

22. A method for the treatment of hypertension comprising administering particulate eplerenone according to claim 11.

23. A method for the treatment of stable patients with left ventricular dysfunction (LVEF≦40%) and clinical evidence of heart failure after recent myocardial infarction to reduce risk of cardiovascular mortality and morbidity, comprising administering particulate eplerenone according to claim 11.

24. A process for preparing a composition comprising eplerenone having a particle size according to claim 11 comprising admixing said eplerenone with one or more pharmaceutically acceptable carriers.

25. A process according to claim 24 wherein the composition is prepared by either wet granulation or dry granulation.

26. A process according to claim 25 comprising:
i) admixing the particulate eplerenone with one or more pharmaceutical excipients;
ii) forming a wet granulation mixture;
iii) granulating mixture;
iv) dry granules;
v) compress granules into a tablet; and
vi) optionally coating the tablet.
